(19) United States
(12) Patent Application Publication
(10) Pub. No.: US 2011/0136916 A1
(43) Pub. Date: Jun. 9, 2011

(54) 6-SHOGAOL FOR USING IN A METHOD FOR THE TREATMENT OF LEUKEMIA

(75) Inventors: Ping Li, Nanjing (CN); Yongbo Peng, Nanjing (CN); Lianwen Qi, Nanjing (CN); Xiaodong Wen, Nanjing (CN); Jiang Ma, Nanjing (CN); Ping Zhou, Nanjing (CN); Lei Zhang, Nanjing (CN); Qun Liu, Nanjing (CN); Ehu Liu, Nanjing (CN); Jun Chen, Nanjing (CN)

(73) Assignee: China Pharmaceutical University, Nanjing (CN)

(21) Appl. No.: 12/938,949

(22) Filed: Nov. 3, 2010

(30) Foreign Application Priority Data
Dec. 8, 2009 (CN) 200910232592.9

(51) Int. Cl. A61K 31/122; C07C 49/255; A61P 35/02

(52) U.S. Cl. 514/678; 568/308

(57) ABSTRACT

The present invention relates to the field of natural drugs, particularly to 6-shogaol for using in a method for the treatment of leukemia. The present invention provides a method for treating leukemia by applying a therapeutically effective dose of 6-shogaolt and this therapeutic method can be used for treating leukemia in mammals including human being.

### CROSS-REFERENCE TO RELATED APPLICATIONS

This application claims priority to CN Application No. 200910232592.9, filed Dec. 8, 2009.

### BACKGROUND OF THE INVENTION

1. Field of the Invention

This invention relates to the field of natural drugs, particularly to 6-shogaol for using in a method for the treatment of leukemia.

2. Description of the Related Art

Ginger and dried ginger are respectively fresh and dry rhizome of Zingiber officinale Rosc. and they are both frequently used in traditional Chinese medicine. Clinical applications of ginger are very extensive and it raises a high degree of concern at home and abroad. Accumulating chemical and pharmacological investigations on ginger and its main active ingredients have been reported in recent years. 6-shogaol (6S for short) is one of the volatile ingredients in ginger, its molecular formula is C₁₇H₂₄O₃ and its molecular weight is 276.37. It is a kind of yellow powder or crystal, which is soluble in ethanol and insoluble in water. Its chemical structural formula is as below:

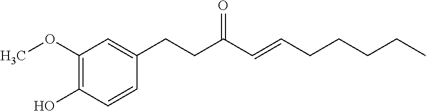

Previous literatures have reported that 6-shogaol has many kinds of biological activities, such as strengthening heart, antiplatelet or anti-thrombus, vasomotion and others. The anti-tumor effects of shogaol ingredients with high activities of ginger have been gradually paid attention in recent years. Recently, a study reported that 6-shogaol had certain anti-tumor activities for human ovarian cancer SK-OV-3 cells, melanoma SK-MEL-2 cells, colon carcinoma HCT15 cells [Cytotoxic components from the dried rhizomes of Zingiber officinale Roscoe. Arch Pharm Res 2008; 31:415-418.] as well as pulmonary carcinoma A549 cells, [6-Shogaol, an Active Constituent of Dietary Ginger, Induces Autophagy by Inhibiting the AKT/mTOR Pathway in Human Non-Small Cell Lung Cancer A549 Cells. J Agric Food Chem. 2009 Oct. 2.], but the activities were not high, and the IC50 for inhibiting the proliferation of A549 cells for 48 hours was about 60 μM; and another reference document reported that 6-shogaol had activities on human colon carcinoma COLO 205 cells [6-Shogaol induces apoptosis in human colorectal carcinoma cells via ROS production, caspase activation, and GADD 153 expression. Mol Nutr Food Res. 2008, 52, 527-37.], but the activities were very weak, and the IC50 for inhibiting the growth of COLO 205 cells was higher than 60 μM.

At the same time, Suekawa M reported that acute toxicity experiment on 6-shogaol in ddY line mice and the LD₅₀ for intravenous injection was about 50.9 mg/kg and the LD₅₀ for intraperitoneal injection was about 109.2 mg/kg, the LD₅₀ for oral administration was 687 mg/kg [Pharmacological studies on ginger. I. Pharmacological actions of pungent constitutents, (6)-gingerol and (6)-shogaol. J Pharmacobiodyn. 1984 Nov.; 7(11):836-48.], indicating that 6-shogaol had excellent safety. Suzanna M. Zick studied the tolerance trial for oral administration with ginger extracts containing 5% total gingerol on healthy volunteers, and the results showed that this kind of compounds can be rapidly absorbed and Tₘₐₓ was reached within about 60 minutes, and it was mainly metabolized in the forms of glucuronide and/or sulfate, the elimination half life was less than two hours and the tolerance dose for single oral administration can be as high as 2 g, and the side effects were significantly lower than the requirements in National Cancer Institute Common Toxicity Criteria (version 2.0) grade 1 [Pharmacokinetics of 6-Gingerol, 8-Gingerol, 10-Gingerol, and 6-Shogaol and Conjugate Metabolites in Healthy Human Subjects. Cancer Epidemiol Biomarkers Prey. 2008; 17(8): 1930-1936. August 2008.], indicating that gingerol compounds were very safe in oral administration and the toxic and side effects of 6-shogaol were low. However, there is no report on the application of this compound in the treatment for preventing leukemia up to now.

### SUMMARY OF THE INVENTION

The present invention disclosed the functions of 6-shogaol in anti-leukemia, and it indicated that 6-shogaol can be used in preparing drugs for anti-leukemia, and its potency was excellent. The present invention provided a method for treating leukemia by applying a therapeutically effective dose of 6-shogaol and this therapeutic method can be used in mammals including human being.

In their research on the pharmacological activities of 6-shogaol, the inventors found that 6-shogaol had excellent therapeutic efficacy in inhibiting leukemic cells. The inventors carried out in vitro MTT screening on human promyelocytic leukemia cell HL-60, human acute T cell leukemic cell Jurkat, human acute myelocytic leukemia cell U937, human chronic granulocytic leukemia K562 and other cells, and they found that 6-shogaol had strong anti-proliferation activities on the above mentioned leukemic cell lines and it showed excellent time-effect and dose-effect relationships, and then they detected the apoptotic rates of Jurkat cells at different concentrations (0, 1, 2.5, 5, 10 and 15 μM) and at different time with the same concentration (15 μM for 0, 2, 4, 6, 12 and 24 hours) by using apoptotic double-staining method (Annexin V/PI) with a flow cytometer. According to the statistic analysis, the results demonstrated that it can significantly induce the apoptosis of human leukemia Jurkat cells and showed excellent time-effect and dose-effect relationships. At the same time, the inventors also applied this compound to peripheral blood lymphocytes from 30 cases of clinical leukemia patients in order to confirm its clinical therapeutic efficacy and anticipate the side effects, and it was found after detecting the apoptosis with the flow cytometer that it can significantly induce the apoptosis of peripheral blood lymphocytes in the leukemia patients, further confirming its excellent anti-leukemia activities. No toxic reaction was found after it was used on normal hepatic cells L-O2. Furthermore, we applied this compound to peripheral blood lymphocytes from 10 cases of healthy volunteers, and after the detection with the flow cytometer, the results showed that no significant difference was found in the induction of apoptosis, indicating that it had slight injuries on peripheral blood lymphocytes from healthy volunteers and the toxicity for 6-shogaol to prevent leukemia was low. It is recommended that the dosage for oral administration in the treatment on leukemia in clinical application could be about 8-50 mg/kg, 1-3 times a day when it is calculated by body weight.

Further illustrations are carried out on the pharmacological activities of 6-shogaol by combining with the following examples.

### BRIEF DESCRIPTION OF THE DRAWINGS

FIG. 1 showed the result of IC₅₀ in MTT detection on the effects of 6-shogaol on different cell lines;

FIG. 2 showed the effects of different concentrations of 6-shogaol on the growth inhibition of HL-60 cell line after 24 hours;

FIG. 3 showed the effects of different concentrations of 6-shogaol on the growth inhibition of Jurkat cell line after 24 hours;

FIG. 4 showed the effects of different concentrations of 6-shogaol on the growth inhibition of U937 cell line after 24 hours;

FIG. 5 showed the effects of different concentrations of 6-shogaol on the growth inhibition of K562 cell line after 24 hours;

FIG. 6 showed the effects of different concentrations of 6-shogaol on the growth inhibition of HL-60 cell line after 72 hours;

FIG. 7 showed the effects of different concentrations of 6-shogaol on the growth inhibition of Jurkat cell line after 72 hours;

FIG. 8 showed the effects of different concentrations of 6-shogaol on the growth inhibition of U937 cell line after 72 hours

FIG. 9 showed the effects of different concentrations of 6-shogaol on the growth inhibition of K562 cell line after 72 hours.

### DETAILED DESCRIPTION OF THE PREFERRED EMBODIMENT

### Embodiment 1

### Anti-Leukemia Effects of 6-Shogaol In Vitro

Cells in logarithmic growth phase were collected and then inoculated in 1˜2×10⁴ cells/well in a 96-well plate respectively according to the sizes of cells and whether they attached to the wall, the cells were centrifuged and the supernatant was removed after growing for 24 hours, and then drug administration was carried out according to the groups as below: the drug group and the drug free group were set up for tumor cells (the concentrations ranged from 1˜20 μM), 5 or 6 duplicates were set for each group and the cells were incubated for 24 or 72 hours. The supernatant was removed and 100 μl serum free culture solution containing 0.5 mg/ml MTT (tetrazolium) was added for further incubation for 4 hours, subsequently 100 μl DMSO (dimethyl sulfoxide) was added and the plate was kept on the micro-shaker for 10 minutes, and the OD value was determined on a micro-plate reader at the wavelength of 570 nm. Evaluations on the toxicity were carried out on normal human cell line LO-2 and each test was repeated for three times. The results were shown in Table 1 and FIG. 1.

TABLE 1
IC50 for the effects of 6-shogaol on different cell lines (n = 3, X̅ ± s)

| Cell line | HL-60 | Jurkat | U937 | K562 |
|---|---|---|---|---|
| Mean IC₅₀ after 24 hours (μM) | 6.93 ± 1.28 | 5.60 ± 0.40 | 7.63 ± 0.47 | 8.50 ± 0.80 |
| Mean IC₅₀ after 72 hours (μM)) | 3.70 ± 0.25* | 3.30 ± 0.30* | 4.93 ± 0.70* | 5.00 ± 0.62* |

Notice:
*indicated the comparison of IC50 at 72 hours with that at 24 hours, p < 0.01.

It can be seen from Table 1 and FIG. 1 that the IC50 of 6-shogaol on human promyelocytic leukemia cell HL-60, human acute T cell leukemic cell Jurkat, human acute myelocytic leukemia cell U937 and human chronic granulocytic leukemia K562 after 24 hours were lower than 10 μM and their 1050 were lower than 5 μM after 72 hours, indicating that 6-shogaol has relatively satisfactory effects in preventing the proliferation of HL-60, Jurkat, U937 and K562 cells.

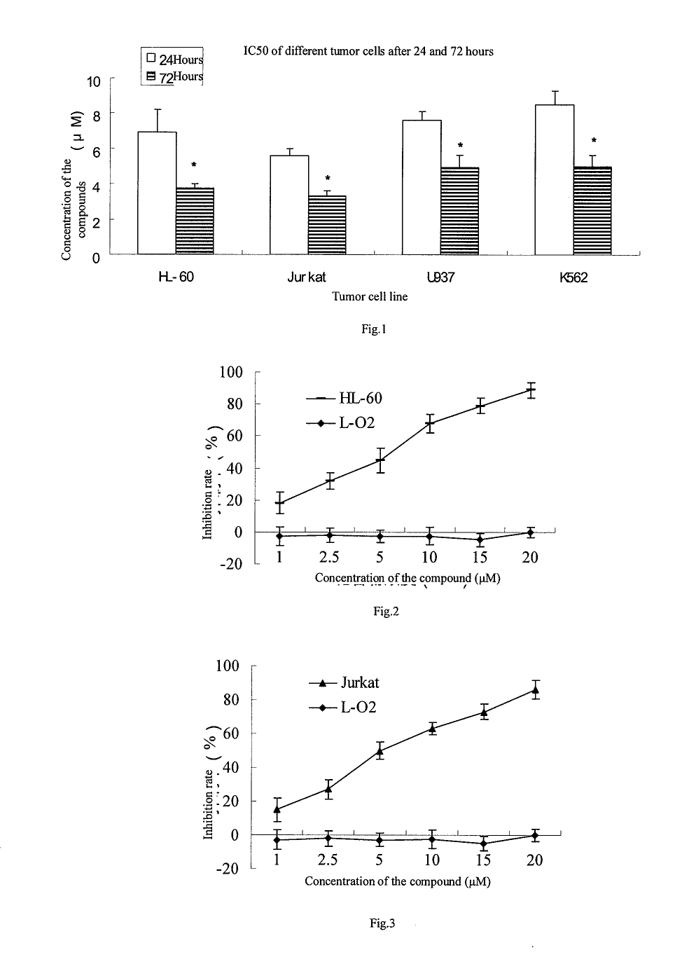

FIG. 2 showed the growth inhibition on HL-60 and LO-2 cell lines after 24 hours at different concentrations of the compound (1, 2.5, 5, 10, 15 and 20 μM), and the results showed that the activities of HL-60 cells significantly decreased with the increase in the concentration of the compound in comparison to the control group without the compound, indicating that 6-shogaol can inhibit the proliferation of tumor cells in a concentration dependent manner; while it didn't inhibit the proliferation of normal hepatic cell line LO-2.

FIG. 3 showed the growth inhibition on Jurkat and LO-2 cell lines after 24 hours at different concentrations of the compound (1, 2.5, 5, 10, 15 and 20 μM), and the results showed that the activities of Jurkat cells significantly decreased with the increase in the concentration of the compound in comparison to the control group without the compound, indicating that 6-shogaol can inhibit the proliferation of tumor cells in a concentration dependent manner; while it didn't inhibit the proliferation of normal hepatic cell line LO-2.

FIG. 4 showed the growth inhibition on U937 and LO-2 cell lines after 24 hours at different concentrations of the compound (1, 2.5, 5, 10, 15 and 20 μM), and the results showed that the activities of U937 cells significantly decreased with the increase in the concentration of the compound in comparison to the control group without the compound, indicating that 6-shogaol can inhibit the proliferation of tumor cells in a concentration dependent manner; while it didn't inhibit the proliferation of normal hepatic cell line LO-2.

FIG. 5 showed the growth inhibition on K562 and LO-2 cell lines after 24 hours at different concentrations of the compound (1, 2.5, 5, 10, 15 and 20 μM), and the results showed that the activities of K562 cells significantly decreased with the increase in the concentration of the compound in comparison to the control group without the compound, indicating that 6-shogaol can inhibit the proliferation of tumor cells in a concentration dependent manner; while it didn't inhibit the proliferation of normal hepatic cell line LO-2.

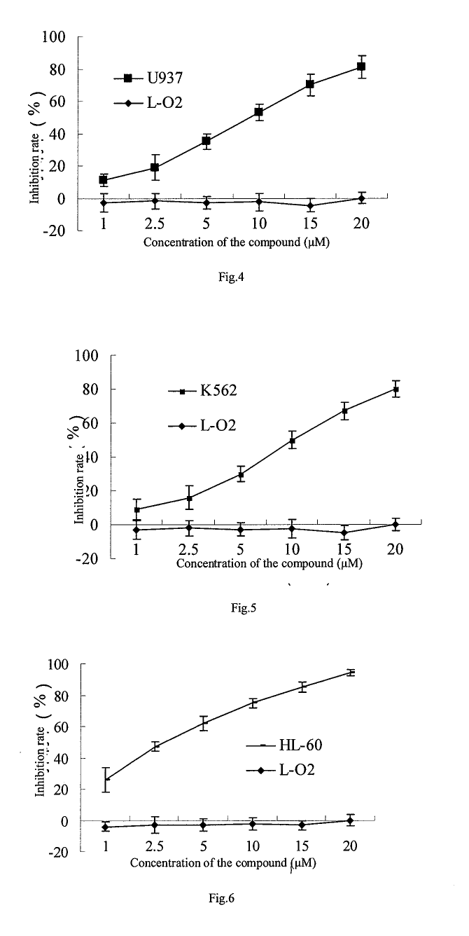

FIG. 6 showed the growth inhibition on HL-60 and LO-2 cell lines after 72 hours at different concentrations of the compound (1, 2.5, 5, 10, 15 and 20 μM), and the results showed that the activities of HL-60 cells significantly decreased with the increase in the concentration of the compound in comparison to the control group without the compound, indicating that 6-shogaol can inhibit the proliferation of tumor cells in a concentration dependent manner; while it didn't inhibit the proliferation of normal hepatic cell line LO-2.

FIG. 7 showed the growth inhibition on Jurkat and LO-2 cell lines after 72 hours at different concentrations of the compound (1, 2.5, 5, 10, 15 and 20 μM), and the results showed that the activities of Jurkat cells significantly decreased with the increase in the concentration of the compound in comparison to the control group without the compound, indicating that 6-shogaol can inhibit the proliferation of tumor cells in a concentration dependent manner; while it didn't inhibit the proliferation of normal hepatic cell line LO-2.

FIG. 8 showed the growth inhibition on U937 and LO-2 cell lines after 72 hours at different concentrations of the compound (1, 2.5, 5, 10, 15 and 20 μM), and the results showed that the activities of U937 cells significantly decreased with the increase in the concentration of the compound in comparison to the control group without the compound, indicating that 6-shogaol can inhibit the proliferation of tumor cells in a concentration dependent manner; while it didn't inhibit the proliferation of normal hepatic cell line LO-2.

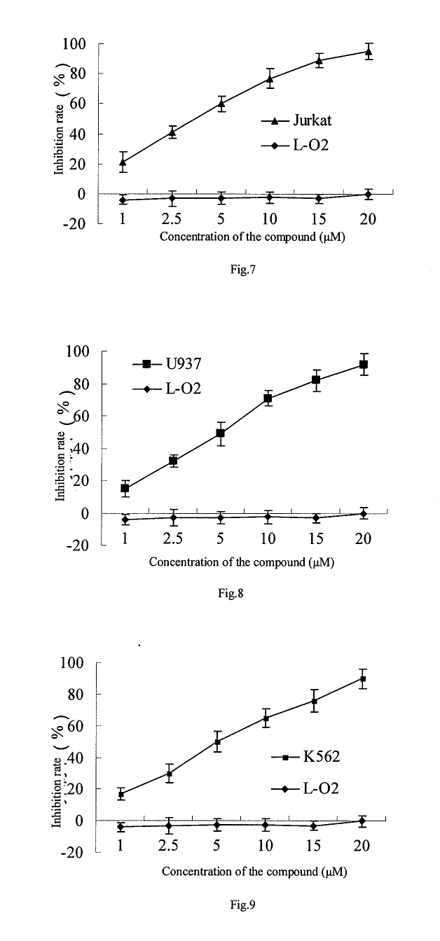

FIG. 9 showed the growth inhibition on K562 and LO-2 cell lines after 72 hours at different concentrations of the compound (1, 2.5, 5, 10, 15 and 20 μM), and the results showed that the activities of K562 cells significantly decreased with the increase in the concentration of the compound in comparison to the control group without the compound, indicating that 6-shogaol can inhibit the proliferation of tumor cells in a concentration dependent manner; while it didn't inhibit the proliferation of normal hepatic cell line LO-2.

From the above-mentioned experimental results, it can be seen that 6-shogaol had excellent effects in inhibiting the proliferation of HL-60, Jurkat, U937 and K562 cells as well as excellent time-effect and dose-effect relationships, while it has low toxicity on normal hepatic cell line LO-2.

### Embodiment 2

### Detection for the Effects of 6-Shogaol on the Apoptosis of Human Leukemia Jurkat Cells

Jurkat cells in logarithmic growth phase of growth were collected (3−10×10⁵) and inoculated to a 24-well plate in 3×10⁵ cells/mL/well, then after incubation for 24 hours, different concentrations of 6-shogaol (0, 1, 2.5, 5, 10 and 15 μM) were added for treatment for 24 hours and also 6-shogaol at a concentration of 15 μM were added for treatment at different durations (0, 2, 4, 6, 12 and 24 h). After reacting with 6-shogaol under different conditions, the cells were collected into 1 mL centrifuge tubes and then centrifuged at 500 rpm (or 100×g) for 5 minutes, the supernatant was removed and the cells were rinsed with 0.01 M PBS at 500 rpm (or 100×g) for 5 minutes twice, and the flow cytometry analysis (Becton Dickinson FACScan Flow Cytometer) was carried out after adding binding solution and staining according to the directions for use of Annexin V/PI double staining kit (BD Company). The FACS data were analyzed by professional operators of the flow cytometer by using FLOWJO software (Tree Star, Calif.) software, the apoptotic rate was counted by the sum of early apoptosis and late apoptosis, and the results were shown in Table 2 and Table 3. After t-test, the results of the statistical analysis showed that 6-shogaol can significantly induce the apoptosis of leukemic Jurkat cells and it showed concentration dependent and time dependent manners, indicating that the inducing apoptosis of leukemic cells was one of the mechanisms for 6-shogaol to inhibit the proliferation of leukemic cells.

TABLE 2
Results for the detection on the effects of different concentrations of 6-shogaol on the
apoptosis of Jurkat cells (n = 3, X̅ ± s)

| | Concentration (μM) | | | | | |
|---|---|---|---|---|---|---|
| | 0 | 1 | 2.5 | 5 | 10 | 15 |
| Apoptotic rate (%) | 5.0 ± 1.4 | 11.2 ± 0.9bd | 17.8 ± 1.4bd | 25.9 ± 3.2bc | 48.7 ± 3.1bd | 73.2 ± 2.8bd |

Notice:
a showing the result in comparison to the control cell group, p < 0.05;
b showing the result in comparison to the control cell group, p < 0.01;
c showing the result in comparison to that of the previous concentration or previous time point, p < 0.05;
d showing the result in comparison to that of the previous concentration or previous time point, p < 0.01.

TABLE 3
Results for the detection on the effects of 15 μM 6-shogaol at different time points on the
apoptosis of Jurkat cells (n = 3, X̅ ± s)

| | Time (hour) | | | | | |
|---|---|---|---|---|---|---|
| | 0 | 2 | 4 | 6 | 12 | 24 |
| Apoptotic rate (%) | 5.9 ± 0.8 | 7.7 ± 2.1 | 14.6 ± 2.5ac | 28.5 ± 3.3bd | 45.3 ± 2.6bd | 72.3 ± 2.4bd |

Notice:
a showing the result in comparison to the control cell group, p < 0.05;
b showing the result in comparison to the control cell group, p < 0.01;
c showing the result in comparison to that of the previous concentration or previous time point, p < 0.05;
d showing the result in comparison to that of the previous concentration or previous time point, p < 0.01.

### Embodiment 3

### Effects of 6-Shogaol, on Peripheral Blood Lymphocytes of Clinical Leukemia Patients

After application by the research group and approval by the Ethics Committee of the hospital, the leukemia patients (healthy volunteers) signed the informed consent and their fresh blood was collected and transferred into heparin anticoagulation tubes, then the same volume of serum free D-Hank buffer (preheated at 37° C.) was added to re-suspend the cells, and the suspension was added to the pre-laid human lymphocyte separating medium (preheated at 37° C.), and the volume ratio between the lymphocyte separating medium and the cell suspension should be no lower than 1:1, and the mixture was centrifuged at room temperature (20-30° C.) at 500×g by a horizontal centrifuge for 30 minutes. The upper layer plasma was removed and the white fog layer in the middle was carefully collected and transferred to 5 mL (or 1-2 times of the volume) PBS (or serum free buffer for cell culture), then it was centrifuged at 200×g or 1000 rpm for 10 minutes at room temperature (20-30° C.), and the supernatant was removed, and the obtained precipitation was the peripheral blood lymphocytes (PBMC). The cells were rinsed twice according to the above-mentioned operations at 500×g by the horizontal centrifuge for 10 minutes at room temperature (20-30° C.), subsequently the cells were collected and their cell vitality was detected by using dye exclusion method for living cells (Trypan blue).

The cells were added to a 24-well plate in 3×10⁵ cells/mL/well and different concentrations of 6-shogaol (10 and 20 μM) for treatment for 24 hours after they were incubated for 24 hours. Negative control group (the group without drug) and the treatment group with 6-shogaol were set up for PBMCs from different individuals respectively. After the cells were treated with 6-shogaol for 24 hours, they were transferred into 10 mL glass centrifuge tubes and centrifuged at 500 rpm (or 100×g) for 5 minutes to remove the supernatant. The cells were then rinsed with 0.01 M PBS at 500 rpm (or 100×g) for 5 minutes twice. Subsequently, after adding binding solution and staining according to the directions for use of Annexin V/PI double staining kit (BD Company), flow cytometry analysis (Becton Dickinson FACScan Flow Cytometer) was carried out. The FACS data were analyzed by professional operators of the flow cytometer by using FLOWJO software (Tree Star, Calif.) software, the apoptotic rate was counted by the sum of early apoptosis and late apoptosis, and the results were shown in Table 4 and Table 5. After t-test, the results of the statistical analysis showed that 6-shogaol can significantly induce the apoptosis of PBMC cells in the 30 cases of leukemia patients and had no significant effects on the apoptosis of PBMC cells from 10 cases of healthy volunteers, indicating that inducing apoptosis of leukemic cells was one of the mechanisms for 6-shogaol to inhibit the proliferation of leukemic cells.

TABLE 4
Results for the detection of PBMC apoptosis by 6-shogaol in healthy volunteers (n = 3, X̅ ± s)

| | Healthy volunteers (N) | | | | | | | | | |
|---|---|---|---|---|---|---|---|---|---|---|
| | N1 | N2 | N3 | N4 | N5 | N6 | N7 | N8 | N9 | N10 |
| Control apoptotic rate (%) | 18.5 ± 1.7 | 10.2 ± 2.7 | 20.8 ± 1.4 | 7.9 ± 1.7 | 14.5 ± 3.7 | 13.2 ± 4.7 | 13.8 ± 2.4 | 16.9 ± 2.7 | 13.7 ± 1.2 | 10.5 ± 2.7 |
| Apoptotic rate of 10 μM | 17.5 ± 0.8 | 13.6 ± 0.9 | 27.6 ± 2.4 | 8.5 ± 0.8 | 16.6 ± 4.8 | 14.6 ± 0.9 | 14.6 ± 1.4 | 18.5 ± 1.8 | 12.5 ± 2.9 | 12.9 ± 1.3 |
| 6-shogaol (%) | | | | | | | | | | |
| Apoptotic rate of 20 μM | 22.8 ± 0.6 | 12.3 ± 1.4 | 31.9 ± 1.7 | 9.7 ± 1.2 | 12.8 ± 3.6 | 12.3 ± 1.4 | 15.9 ± 0.7 | 21.7 ± 1.6 | 14.8 ± 0.9 | 16.5 ± 0.7 |
| 6-shogaol (%) | | | | | | | | | | |

Notice:
a showing the result in comparison to the negative control group, p < 0.01.

TABLE 5
Result 1 for the detection of PBMC apoptosis by 6-shogaol in
healthy volunteers (n = 3, X̅ ± s)

| | Patients (P) | | | | |
|---|---|---|---|---|---|
| | P1 | P2 | P3 | P4 | P5 |
| Control apoptotic | 6.5 ± 2.3 | 9.2 ± 1.8 | 12.8 ± 2.7 | 9.4 ± 3.1 | 17.5 ± 2.5 |
| rate (%) | | | | | |
| Apoptotic rate of 10 μM | 38.5 ± 3.7a | 27.6 ± 1.3a | 47.9 ± 1.6a | 35.7 ± 3.8a | 40.5 ± 6.1a |
| 6-shogaol (%) | | | | | |
| Apoptotic rate of 20 μM | 56.9 ± 2.2a | 50.1 ± 5.4a | 72.9 ± 3.1a | 69.7 ± 1.6a | 61.9 ± 4.2a |
| 6-shogaol (%) | | | | | |
| | Patients (P) | | | | |
| | P6 | P7 | P8 | P9 | P10 |
| Control apoptotic | 13.2 ± 4.7 | 17.9 ± 1.9 | 11.8 ± 1.7 | 12.9 ± 2.2 | 14.9 ± 3.7 |
| rate (%) | | | | | |
| Apoptotic rate of 10 μM | 29.3 ± 1.7a | 44.6 ± 3.8a | 31.3 ± 2.7a | 27.5 ± 2.9a | 42.4 ± 3.1a |
| 6-shogaol (%) | | | | | |
| Apoptotic rate of 20 μM | 52.7 ± 2.3a | 75.9 ± 1.7a | 64.7 ± 2.4a | 44.8 ± 3.9a | 73.5 ± 3.7a |
| 6-shogaol (%) | | | | | |

Notice:
a showing the result in comparison to the negative control group, p < 0.01;
b showing the result in comparison to the 10 μM 6-shogaol group, p < 0.05;
c showing the result in comparison to the 10 μM 6-shogaol group, p < 0.01.

TABLE 5
Result 2 for the detection of PBMC apoptosis by 6-shogaol in healthy volunteers (n = 3, X̅ ± s)

| P11 | P12 | P13 | P14 | P15 | P16 | P17 | P18 | P19 | P20 |
|---|---|---|---|---|---|---|---|---|---|
| 12.5 ± 2.8 | 19.8 ± 3.6 | 22.4 ± 3.7 | 14.4 ± 2.5 | 19.1 ± 1.5 | 23.2 ± 1.5 | 17.9 ± 1.9 | 16.8 ± 2.7 | 24.9 ± 3.1 | 30.9 ± 2.1 |
| 29.7 ± 2.4a | 49.3 ± 4.1a | 55.9 ± 2.7a | 26.7 ± 1.9a | 34.5 ± 3.1a | 39.3 ± 2.3a | 44.6 ± 3.8a | 33.1 ± 3.7a | 37.9 ± 4.9a | 52.9 ± 2.7a |
| 50.1 ± 3.3ac | 80.8 ± 5.1ac | 82.9 ± 2.5ac | 39.7 ± 2.6ac | 47.9 ± 4.2ab | 46.7 ± 1.6ab | 55.9 ± 1.7ab | 54.7 ± 2.6ac | 51.8 ± 7.9ac | 63.6 ± 1.2ab |

Notice:
a showing the result in comparison to the negative control group, p < 0.01;
b showing the result in comparison to the 10 μM 6-shogaol group, p < 0.05;
c showing the result in comparison to the 10 μM 6-shogaol group, p < 0.01.

TABLE 5
Result 3 for the detection of PBMC apoptosis by 6-shogaol in healthy volunteers (n = 3, X̅ ± s)

| P21 | P22 | P23 | P24 | P25 | P26 | P27 | P28 | P29 | P30 |
|---|---|---|---|---|---|---|---|---|---|
| 14.8 ± 1.7 | 7.3 ± 1.5 | 17.2 ± 2.6 | 20.8 ± 1.8 | 30.7 ± 4.4 | 21.7 ± 3.1 | 14.6 ± 2.8 | 22.8 ± 3.1 | 18.9 ± 2.6 | 11.9 ± 2.5 |
| 31.5 ± 4.3a | 20.5 ± 3.1a | 36.2 ± 5.7a | 42.7 ± 2.4a | 47.8 ± 2.9a | 43.3 ± 4.9a | 30.9 ± 1.7a | 39.6 ± 2.2a | 33.7 ± 3.5a | 28.6 ± 2.9a |
| 44.5 ± 2.4ab | 38.5 ± 2.1ac | 67.1 ± 3.7ac | 58.7 ± 3.9ac | 69.1 ± 3.3ac | 56.9 ± 5.2ac | 47.6 ± 3.6ab | 56.3 ± 3.8ac | 42.4 ± 2.9ab | 51.9 ± 4.2ac |

Notice:
a showing the result in comparison to the negative control group, p < 0.01;
b showing the result in comparison to the 10 μM 6-shogaol group, p < 0.05;
c showing the result in comparison to the 10 μM 6-shogaol group, p < 0.01.

What is claimed is:

1. A method of treating leukemia in mammals comprising administering a therapeutically effective dose of 6-shogaol to a mammal in need thereof.

2. The method of claim 1, wherein the mammal is human.

3. A pharmaceutical preparation for preventing or treating leukemia comprising 6-shogaol in a pharmaceutically acceptable carrier.

4. A composition for preventing or treating leukemia comprising 6-shogaol.
